(19) United States
(12) Patent Application Publication
(10) Pub. No.: US 2006/0161101 A1
(43) Pub. Date: Jul. 20, 2006

(54) Surgical system and handpiece

(75) Inventors: Ramon C. Dimalanta, Rancho Santa Margarita, CA (US); Amir H. Khashayar, Laguna Niguel, CA (US); Sean C. Madden, Mission Viejo, CA (US); Glenn Sussman, Laguna Nigel, CA (US); John R. Underwood, Laguna Nigel, CA (US)

Correspondence Address: ALCON RESEARCH, LTD., R&D COUNSEL, Q-148, 6201 SOUTH FREEWAY, FORT WORTH, TX, 76134-2099, US

(73) Assignee: Alcon, Inc.

(21) Appl. No.: 11/037,062

(22) Filed: Jan. 18, 2005

(51) Int. Cl. A61M 31/00

(52) U.S. Cl. 604/67; 606/107

(57) ABSTRACT

A surgical system for a liquefaction handpiece. The system uses a pressure sensing element located on the output side of the fluid pulse engine to read directly the pressure of the fluid pulse exiting the engine.

### BACKGROUND OF THE INVENTION

This invention relates generally to the field of cataract surgery and more particularly to a system and handpiece for practicing the liquefaction technique of cataract removal.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquefies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

Recently, a new cataract removal technique has been developed that involves the injection of hot (approximately 45° C. to 105° C.) water or saline to liquefy or gellate the hard lens nucleus, thereby making it possible to aspirate the liquefied lens from the eye. Aspiration is conducted with the injection of the heated solution and the injection of a relatively cool solution, thereby quickly cooling and removing the heated solution. This technique is more fully described in U.S. Pat. No. 5,616,120 (Andrew, et al.), the entire contents of which is incorporated herein by reference. The apparatus disclosed in the publication, however, heats the solution separately from the surgical handpiece. Temperature control of the heated solution can be difficult because the fluid tubings feeding the handpiece typically are up to two meters long, and the heated solution can cool considerably as it travels down the length of the tubing.

Recently, a device was commercially introduced that practices the liquefaction method. The AQUALASE® handpiece, part of the INFINITI® Vision System available from Alcon Laboratories, Inc., Fort Worth, Texas, produces pulses of heated irrigation fluid for lens removal and cortical cleanup. This device, however, does not monitor the pressure of the pulse directly. Instead, the pressure of the fluid pulses is calculated from the duration of the electrical power pulse supplied to the fluid pulse engine.

Therefore, a need continues to exist for a control system for a liquefaction handpiece that can directly measure the pressure of the fluid pulses generated by the handpiece.

### BRIEF SUMMARY OF THE INVENTION

The present invention improves upon the prior art by providing a control system for a liquefaction handpiece. The system uses a pressure sensing element located on the output side of the fluid pulse engine to read directly the pressure of the fluid pulse exiting the engine. Such a sensor allows for the active feedback control of the drive signal being sent to the handpiece.

Accordingly, one objective of the present invention is to provide a surgical console control system.

Another objective of the present invention is to provide a surgical console control system that generates a control signal indicative of the pressure in a fluid pulse generated in a handpiece connected to the control system.

Another objective of the present invention is to provide a surgical console control system that provides a pressure feedback signal that can be used in adjusting the drive signal for a liquefaction handpiece.

These and other advantages and objectives of the present invention will become apparent from the detailed description and claims that follow.

### BRIEF DESCRIPTION OF THE DRAWINGS

FIG. 1 is a perspective view of a surgical system that can be used with the control system of the present invention.

FIG. 2 is an exploded, partial cross-sectional view of a prior art handpiece.

FIG. 3 is an exploded, partial cross-sectional view of a handpiece embodying the present invention.

### DETAILED DESCRIPTION OF THE INVENTION

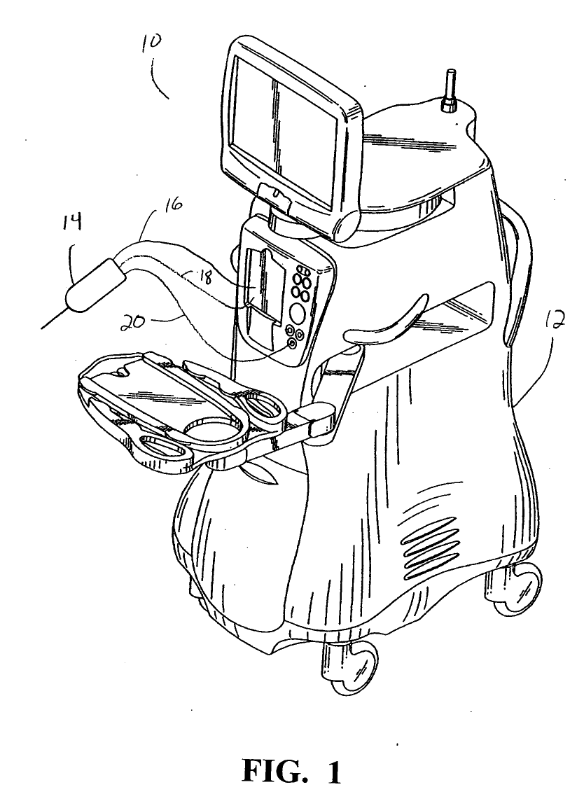

As best seen in FIG. 1, system 10 of the present invention generally includes control console 12 and handpiece 14. System 10 may be any suitable system, such as the INFINITI® Vision System available from Alcon Laboratories, Inc., Fort Worth, Tex. Handpiece 14 may be any suitable handpiece, such as the AQUALASE® handpiece available from Alcon Laboratories, Inc., Fort Worth, Tex. Handpiece 10 is fluidly connected to console 12 by fluid tubings 16 and 18, and electrically connected to console 12 by electrical cable 20, such tubings and cabling being well-known in the art. Control console 12 contains appropriate hardware and software (not shown, but well-known in the art) for providing control signals to handpiece 14.

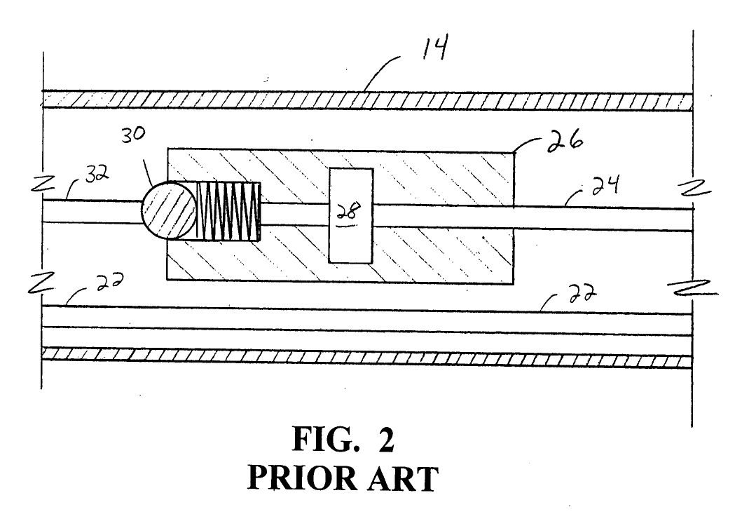

As best seen in FIG. 2, prior art handpieces for practicing the liquefaction technique generally include aspiration line 22, connected to console 12 through tubing 18, irrigation line 24, connected to console 12 by tubing 16. Irrigation line 24 provides sterile irrigation fluid to pulse engine 26. Pulse engine 26 contains boiling chamber 28 that produces the heater fluid pressure pulses in a manner well-known in the art. Irrigation fluid boiled in chamber 28 exits engine 26 through check valve 30 and out through injection tube 32. The pressure of the pulse exiting engine 26 through injection tube 32 is calculated based on the size and duration of the electrical drive signal sent to engine 26 through cable 20 by console 12.

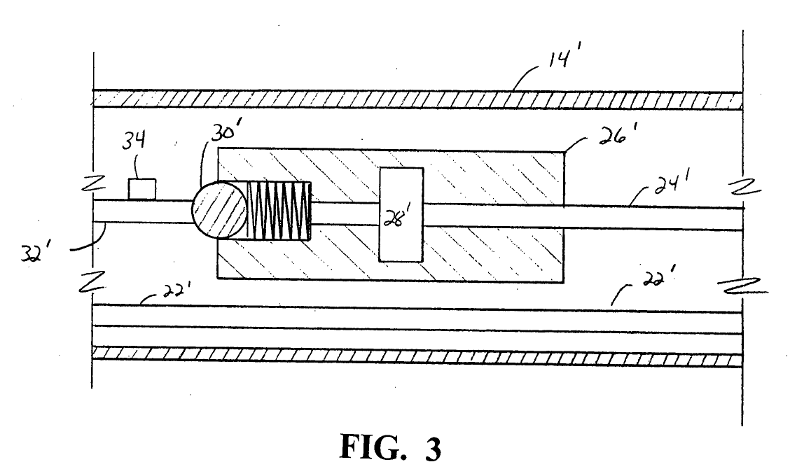

As best seen in FIG. 3, the handpiece of the present invention generally includes aspiration line 22′, connected to console 12 through tubing 18, irrigation line 24′, connected to console 12 by tubing 16. Irrigation line 24′ provides sterile irrigation fluid to pulse engine 26′. Pulse engine 26′ contains boiling chamber 28′ that produces the heater fluid pressure pulses in a manner well-known in the art. Irrigation fluid boiled in chamber 28′ exits engine 26′ through check valve 30′ and out through injection tube 32′. Handpiece 14′ also includes pressure sensing element 34 that is fluidly connected to injection tube 32′ so as to be able to sense or detect the pressure within injection tube 32′. Pressure sensing element 34 may be any suitable pressure sensor cable of directly measuring pressure within injection tube 32′ , such as a piezoelectric pressure sensing element. The pressure of the pulse exiting engine 26′ through injection tube 32′ is not calculated based on the size and duration of the electrical drive signal sent to engine 26 through cable 20 by console 12 as in the prior art system illustrated in FIG. 2, but is directly read by pressure sensing element 34. This pressure information can be used by console 12 in a feedback control loop for regulating the power supplied to engine 26′.

This description is given for purposes of illustration and explanation. It will be apparent to those skilled in the relevant art that changes and modifications may be made to the invention described above without departing from its scope or spirit. For example, it will be recognized by those skilled in the art that the present invention may be combined with ultrasonic and/or rotating cutting tips to enhance performance.

We claim:

1. A liquefaction handpiece, comprising:
a) an aspiration line;
b) an irrigation line;
c) an injection tube;
d) an engine located between the irrigation line and the injection tube, the engine having a pumping chamber that is fluidly connection to the irrigation line and the injection tube; and
e) a pressure sensing element located on and in fluid communication with the injection tube.

2. The handpiece of claim 1 wherein the pressure sensing element is a piezoelectric element.

3. A surgical system, comprising:
a) a control console capable of generating a handpiece control signal;
b) a handpiece having an engine with a pumping chamber, the handpiece fluidly and electrically connected to the console; and
c) a pressure sensing element located in the handpiece, the pressure sensing element in fluid communication with the engine.

4. The system of claim 3 wherein the claim 1 wherein the pressure sensing element is a piezoelectric element.
